(19) United States
(12) Patent Application Publication
(10) Pub. No.: US 2015/0351665 A1
(43) Pub. Date: Dec. 10, 2015

(54) METHOD FOR MEASURING POWER GENERATED DURING RUNNING

(71) Applicant: MedHab, LLC, Mansfield, TX (US)

(72) Inventor: Johnny Ross, Mansfield, TX (US)

(21) Appl. No.: 14/826,691

(22) Filed: Aug. 14, 2015

Related U.S. Application Data
(63) Continuation-in-part of application No. 14/709,541, filed on May 12, 2015.
Application No. 14/709,541 is a continuation-in-part of application No. 14/505,106, filed on Oct. 2, 2014.
Application No. 14/505,106 is a continuation-in-part of application No. 14/217,337, filed on Mar. 17, 2014.
Application No. 14/217,337 is a continuation-in-part of application No. 13/749,665, filed on Jan. 24, 2013.
(60) Provisional application No. 61/800,981, filed on Mar. 15, 2013.
(60) Provisional application No. 61/867,064, filed on Aug. 17, 2013.

(51) Int. Cl. A61B 5/103; A61B 5/00; G01L 5/00

(52) U.S. Cl. CPC A61B 5/1038; G01L 5/0052; A61B 5/6807; A61B 5/7271; A61B 2503/10

(57) ABSTRACT

A method for measuring power generated by a person during running includes the use of a pair of sensor insoles, each having force sensors. The method utilizes at least one computer device for performing the following: determining a reference distance ratio for the person; receiving force data from the plurality of force sensors; calculating a distance run based on a product of the reference distance ratio and a total number of impulses received in the force data; determining total force, and time elapsed during which the force data is received from the force sensors; and calculating and reporting power.

### CROSS-REFERENCE TO RELATED APPLICATIONS

This application for a utility patent is a continuation-in-part of the following previously filed utility patent applications:
application Ser. No. 14/709,541, still pending, filed May 12, 2015;
application Ser. No. 14/505,106, still pending, filed Oct. 2, 2014;
application Ser. No. 14/217,337, still pending, filed Mar. 17, 2014; and
application Ser. No. 13/749,665, still pending, filed Jan. 24, 2013.

Application Ser. No. 13/749,665 is a continuation-in-part of the following previously filed utility patent applications:
application Ser. No. 13/741,294, now abandoned, filed Jan. 14, 2013; and
application Ser. No. 13/070,649, now U.S. Pat. No. 8,384,551, filed Mar. 24, 2011.

Application Ser. No. 14/505,106 claims the benefit of the U.S. Provisional Application No. 61/889,878, filed Oct. 11, 2013.

Application Ser. No. 14/217,337 claims the benefit of the following U.S. Provisional applications:
application No. 61/800,981, filed Mar. 15, 2013; and
application No. 61/867,064, filed Aug. 17, 2013.

### BACKGROUND OF THE INVENTION

1. Field of the Invention

This invention relates generally to sensor devices, and more particularly to systems and methods for measuring power generated during running

2. Description of Related Art

Various devices have been developed for measuring power and related metrics generated during a user performing a physical activity. Typical systems utilize accelerometers, gyro meters, and the like for tracking and/or measuring various parameters related to the physical activity.

One example of such a system is shown in Templeman, U.S. Pat. No. 8,744,783, which teaches a system and method for calculating running power that includes accelerometers and force sensors mounted in a shoe. The accelerometers are used to determine distance travelled, and the system relies upon reference to a database of foot force wave forms to determine the style of the person's movement, for making the necessary force computations.

There are various other devices in the prior art that teach sensor devices for measuring force on a user's foot, usually for the purposes of assisting in rehabilitation of a user's leg following an injury, surgery, or similar situation.

One conventional approach discloses a slipper that includes a fluid chamber that enables weight sensing by a load monitor. When not enough weight is applied, or when too much weight is applied, a beeping sound is emitted to guide the patient in rehabilitating an injured leg. Another conventional approach discloses an insertable sole that includes plates having force sensors for determining a load placed upon the sole by a user. An amplifier and AC/DC converter generate a force signal that is received by a processor for generating audible and visual feedback via a piezo-beeper and display screen. Yet another conventional approach teaches a force monitoring shoe utilizing a force monitoring device to measure force exerted on the shoe, warn the patient (e.g., a beeper) if predetermined force levels are exceeded, and collect the accumulated data in a data gathering device. The force sensor may be a resistive sensor pad, and the patient alerting elements may include a wireless transmitter that transmits a signal to a separate unit that vibrates in response to exceeding recommended forces. The data gathering device may be a recorder, or a receiver in a doctor's office. Still another conventional approach discloses a rehabilitation device that measures force exerted on a sensor in a shoe for the purposes of guiding a patient in placing the correct amount of weight on an injured leg. Additionally, many existing sensor devices include accelerometers and other apparatuses for various purposes. For example, a shoe having a built-in electronic wear indicator device that includes an accelerometer for measuring foot movement.

### SUMMARY OF THE INVENTION

The present invention teaches certain benefits in construction and use which give rise to the objectives described below.

One embodiment of the present disclosure includes a method for measuring power generated by a person during running The method includes the use of a pair of sensor insoles, each having a plurality of force sensors. The system includes at least one computer device for performing the following: determining a reference distance ratio for the person that is an approximate distance travelled for each step taken by that person; receiving force data from the plurality of force sensors, the force data including a number of impulses received that correspond to the number of steps taken by the person, and a force for each of the impulses; calculating a distance run based on a product of the reference distance ratio and the total number of impulses received in the force data; calculating a total force based on a sum of the force measured in all of the impulses in the force data; determining time elapsed during which the force data is received from the force sensors; calculating power based on the calculated distance, the elapsed time, and the force data received; and reporting the power determined.

A primary objective of the present invention is to provide a method for measuring power generated by a person during running having advantages not taught by the prior art.

Another objective is to provide a method that is simple to implement and enables the reliable calculation of power generated by a person while running

Another objective is to provide a method for determining power so that a person is better able to monitor and control his or her power during running or similar training, to optimize his or her speed and endurance.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The advantages and features of the present invention will become better understood with reference to the following more detailed description taken in conjunction with the accompanying drawings in which:

FIG. 1 is a top plan view of a sensor insole, according to one embodiment of the present invention;

FIG. 2 is a perspective view of a felt layer on which is mounted two sensor assemblies;

FIG. 3 is a sectional view of a mold in which the felt layer and the sensor assemblies are placed;

FIG. 4 is an exploded perspective view of a sensor sheet removed from the mold once urethane has been injected to form a urethane layer, illustrating the sensor insoles being cut from the sensor sheet;

FIG. 5 is a perspective view of a portable electronic device having a monitoring app installed thereupon for monitoring the movement of a user, and for illustrating the movements of the user on a display of the portable electronic device;

FIG. 6 is a block diagram of the operable components of the portable electronic device of FIG. 5;

FIG. 7 is a perspective view of the portable electronic device having the monitoring app installed thereupon for monitoring the forces measured by force sensors in the sensor insoles and illustrating the data in the form of a pie graph;

FIG. 8 is a perspective view of the portable electronic device having the monitoring app installed thereupon for monitoring the forces measured by the force sensors in the sensor insoles and illustrating the data in the form of a contour plot;

FIG. 9 is a block diagram of one embodiment of a sensor system that includes the portable electronic device, a monitoring computer, and a remote computer for monitoring the sensor system and storing data;

FIG. 10 is a flow diagram illustrating an exemplary method implemented by the portable electronic device of FIG. 9 for being calibrated to measure the power generated by the user while running; and

FIG. 11 is a flow diagram illustrating an exemplary method implemented by the portable electronic device of FIG. 9 for measuring power generated by the user while running

### DETAILED DESCRIPTION OF THE INVENTION

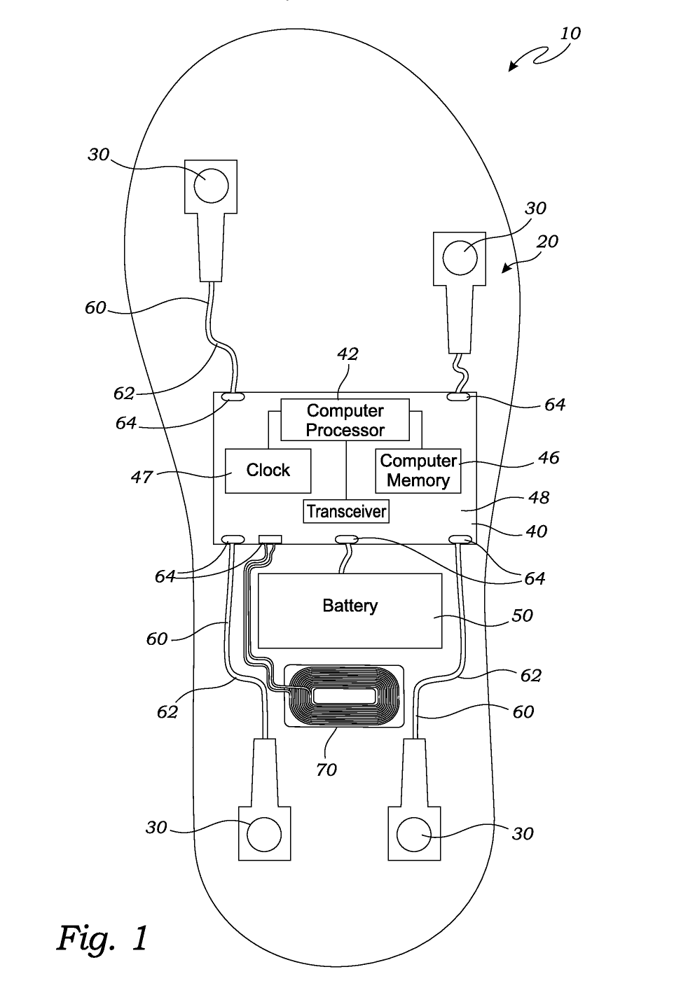

FIG. 1 is a top plan view of a sensor insole 10, as used in one embodiment of the present invention. A felt layer, discussed below, is removed in this view to more clearly show a sensor assembly 20 that is located within the sensor insole 10. Electronic components of the sensor insole 10 are shown in a block diagram to more clearly illustrate the invention.

As illustrated in FIG. 1, the sensor insole 10 is shaped and adapted to fit within a shoe (not shown) of a user, or otherwise positioned against the underside of the foot of the user. A sensor assembly 20 is included in the sensor insole 10 for monitoring various forces and conditions of the sensor insole 10. In this embodiment, the sensor assembly 20 includes force sensors 30. In the embodiment of FIG. 1, the sensor insole 10 may include a printed circuit board (“PCB”) 40 having (or being operably attached to a computer processor 42, a computer memory 46, a battery 50, and the force sensors 30. The force sensors 30 may be any form of sensors useful for sensing force that are known in the art. While four of the force sensors 30 are illustrated, in different embodiments other numbers of the force sensors 30 may be used, depending upon the requirements of the user.

The force sensors 30 are adapted to send signals to the processor 42, transferring the values of the properties sensed by the force sensors 30 individually. Each of the plurality of force sensors 30 may be operably connected to the processor 42 by electrical connectors 60, in this case wires, or any other operative connection known in the art.

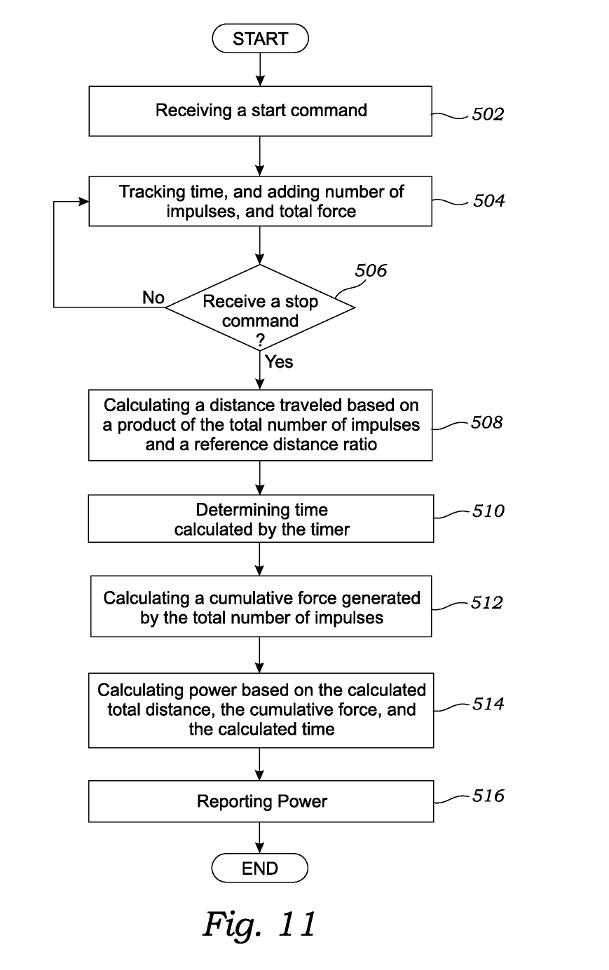

In this embodiment, the wires 60 may be attached to the PCB 40 via soldering; however, the wires 60 may be attached using any techniques or attachment mechanisms known in the art. The solder joints may also be covered with a protective layer, to strengthen the connection to withstand the stresses and strains placed upon the wires 60. This is further discussed in the descriptions of FIGS. 11-13.

The wires 60 may be positioned in an S-curve configuration 62 between the force sensor 30 and the PCB 40. The S-curve configuration 62 provides strain relief during use, so that the electrical connection is not broken during use. For purposes of this application, the term “S-curve configuration” is defined to include any configuration in which the wires 60 are bent in places, so that the wires 60 are long enough to accommodate forces against the various components while in use without breaking any solder joints.

The computer processor 42 and the computer memory 46 may be any form of processor or processors, memory chip(s) or devices, microcontroller(s), and/or any other similar processing devices known in the art.

The battery 50 supplies power to the processor 42 and the plurality of force sensors 30 (and any other components). The battery 50 may be rechargeable which can be charged by an external power source, or in alternative embodiments it may be replaceable. The sensor assembly 20 may further include an inductive charging coil 70 which may be operably mounted adjacent the battery 50 and/or the PCB 40. The inductive charging coil 70 is used to charge the battery 50 by using an external inductive charger (not shown). Other devices or systems known in the art for supplying power may also be utilized, including various ports for charging the battery 50, and/or generating power directly using piezoelectric, solar, or other devices known in the art.

In the present embodiment, the force sensors 30 are piezoresistance based, meaning that the resistance of the circuit in which they have been integrated changes in response to the applied force. Other methods known to those skilled in the art may also be used to provide a force sensing mechanism. The applied force may then be determined by incorporating the force sensors 30 in a voltage divider, whereby the voltage across the force sensor 30 would change in response to the applied force, an RC circuit whereby the time constant would change in response to the applied force, or integrating an Ohmmeter to measure the resistance directly, or other methods of reading the applied force known to those skilled in the art. If a force measurement is desired instead, the known area of measurement allows that to be determined directly. The force sensors 30 have an upper limit to the force they may measure and still be accurate or without breaking Using the plurality of force sensors 30 as shown in the present embodiment allows total force to be shared amongst the force sensors 30 and to measure the force distribution in the user's foot. The use of small sensors allows the force to be sampled over a smaller fraction of the surface area of the foot, giving a proportionally smaller force.

The force sensors 30, in the present embodiment, have a high sampling rate, up to 200 kHz, which is far beyond what would normally be needed for an activity like walking, but may be desirable when one wishes to analyze more impulsive forces, such as those due to running or kicking. In some embodiments, the sampling rate and duration may be adjusted by the user based on the intended application. In some other embodiments, temperature sensors (not shown) may be incorporated into the sensor assembly 20 for providing temperature data. This may be important as the force sensor 30 may also be weakly temperature dependent and therefore changes in temperature may need to be corrected for.

The processor 42 may also include the memory 46 to store data collected by the plurality of sensors 30, and a transceiver 48 to transmit and receive signals for communication between the processor 42 and external computing devices enabled to send and receive the signals. The processor 42, the memory 46 and the transceiver 48 may all be mounted on the PCB 40, or in other suitable locations as determined by one skilled in the art.

The sensor insole 10 may be used in conjunction with a shoe (not shown), including any form of sneaker, slipper, or any other footwear known in the art for holding the insole 10 in mechanical communication with the underside of the person's foot. As a person wearing the shoe runs, force is exerted on the sensor insole 10, and data from the force sensors 30 can be collected. The data collected by the processor 42 from different force sensors 30 may be used in a variety of ways. The sensor assembly 20 may use the transceiver 48 to connect and transfer data from the sensor assembly 20 to a local and/or remote computer (not shown). The data may be transmitted by the transceiver 48 by any number of methods known to those skilled in the art, however, in particular, the data may be transferred in packets or bundles, containing multiple bytes or bits of information. The bundling of the data may be performed according to those skilled in the art for optimizing the data transfer rate between the sensor insole 10 and any remote receiver. Alternatively in another embodiment, the data may be reported via a reporting device worn by the user, attached to the shoe, located nearby, or located remotely. In another embodiment, the data may also be used to compare with a threshold value and take a predefined action based on the comparison. The data may be received, collected, reviewed, and utilized using different forms of computer devices.

The sensor insole 10 may further include a clock 47 for tracking time, or it may be operably connected to another device for this purpose. The function of the clock 47 is discussed in greater detail below.

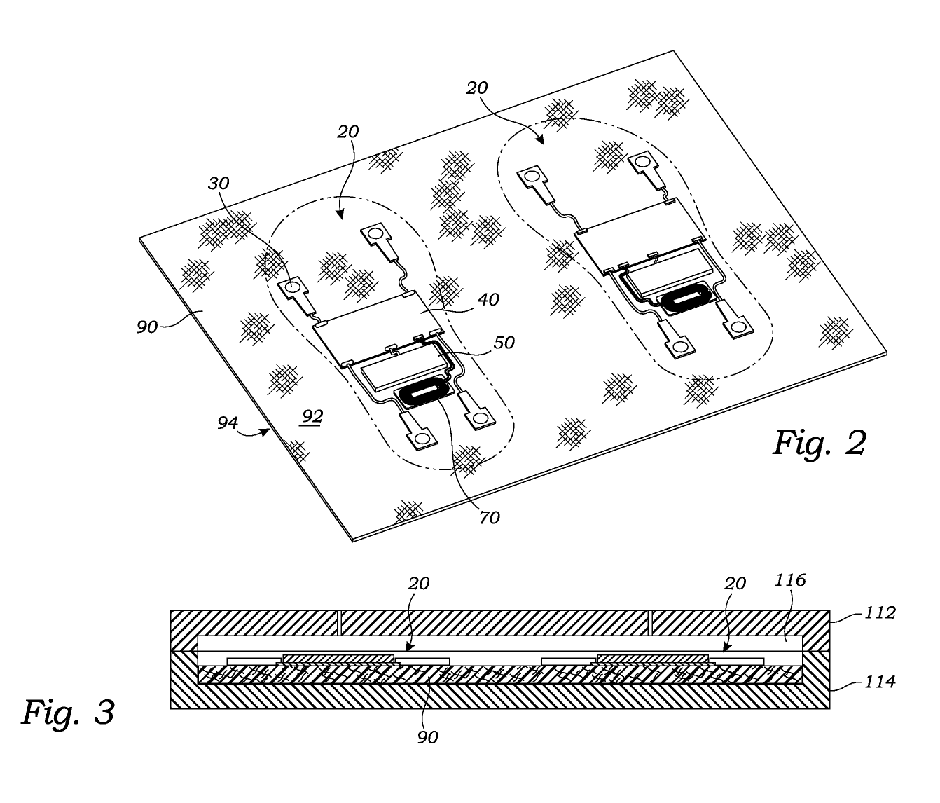

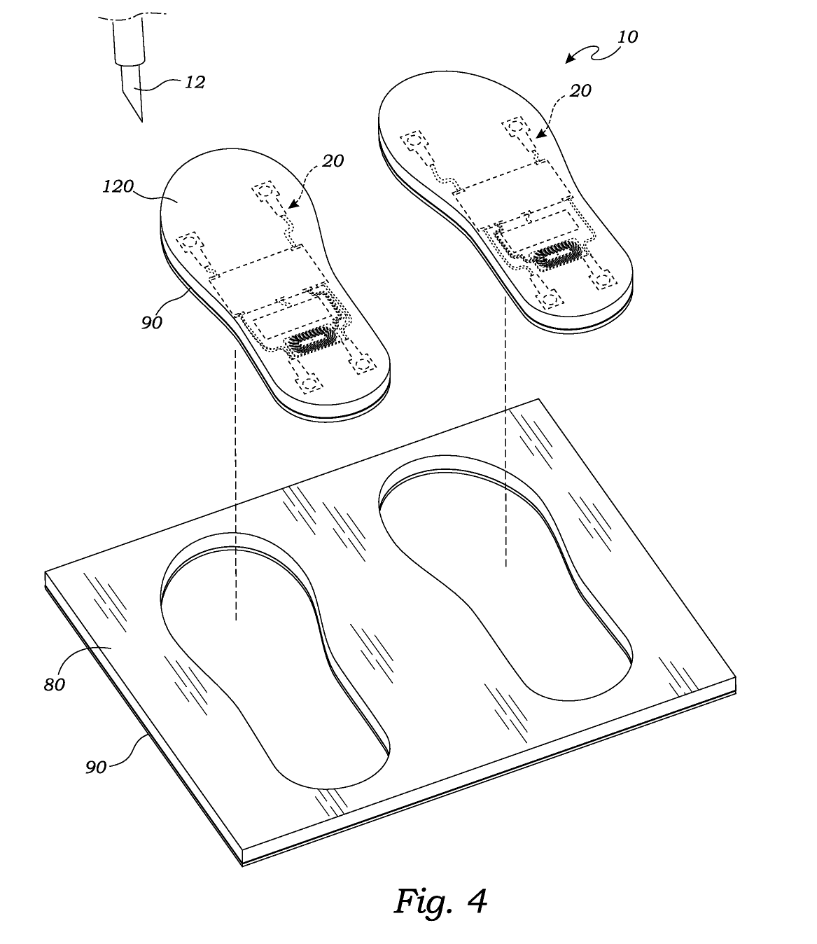

FIGS. 2-4 illustrate one embodiment of how the sensor insoles 10 may be manufactured. FIG. 2 is a perspective view of a felt layer 90 on which is mounted two of the sensor assemblies 20 of FIG. 1. FIG. 2 illustrated one method of manufacturing the sensor insole 10 of FIG. 1. Further steps in the manufacturing process are shown in FIGS. 3 and 4, as discussed in greater detail below.

As illustrated in FIG. 2, the felt layer 90 has a top surface 92 and a bottom surface 94. The felt layer 90 may be large enough for one sensor assembly 20; or alternatively, it may be large enough for a pair of the sensor assemblies 20, as illustrated, or it may be large enough for a larger number of the sensor assemblies 20, depending upon the manufacturing requirements of the user. The felt layer 90 should neither be very thick, such that the force sensors 30 are not able to sense the wearer's foot properties correctly, nor be very thin so that the sensor assembly 20 causes pain or discomfort to the user's foot.

The term “felt layer” is hereby defined to include one or more layers of woven and/or nonwoven material (which may be produced by, e.g., matting, condensing and pressing woolen fibers bonded together by chemical, mechanical, heat or solvent treatment), and to also include one or more layers any form of cloth, flexible synthetic material, and any other layer of material that is suitable for insertion into a shoe consistent with the description of the present invention. The scope of this term should be broadly construed to include any material or materials that may be devised by one skilled in the art for this purpose. The felt layer 90 should be flexible enough to bend as a person wearing the shoe runs, to limit any discomfort felt by the wearer while running

The sensor assemblies 20 may be mounted on the felt layer 90 and fastened in place, or they may just be placed thereupon. In one embodiment, the sensor assembly 20 may be attached to the felt layer 90 using an adhesive (not shown) or a suitable tacky substance. The purpose of attaching the sensor assembly 20 with the felt layer 90 is to retain the location of the force sensors 30 and other components of the sensor assembly 20, such as the PCB 40, the battery 50, and the inductive charging coil 70, during the molding process. Any alternative method which serves the purpose of properly positioning the sensor assembly 20 may also be used and may not require any bonding or direct attachment of the sensor assembly 20 to the felt layer 50, in an alternative embodiment.

FIG. 3 is a sectional view of a mold 110 in which the felt layer 90 and the sensor assemblies 20 may be placed. As illustrated in FIG. 3, the mold 110 may include a top portion 112 and a bottom portion 114 that close together to form a planar internal cavity 116; however, any suitable construction functional as described may be used, according to the knowledge of one skilled in the art. The mold 110 further includes components (not shown) to supply a suitable resilient material (e.g., urethane foam, rubber, or any suitable resilient material known in the art) to form a resilient sheet on top of the felt layer 90 inside the internal cavity 116. The mold 110 may include conduits 117 for injecting the urethane foam and to allow air and gases to escape from the closed mold 110. While one embodiment of a mold, jig, or similar tool is shown, the sensor insole 10 (of FIG. 1) may be manufactured using any similar or equivalent tool or method known in the art, and such alternatives should be considered within the scope of the present invention.

FIG. 4 is an exploded perspective view of a sensor sheet 80 removed from the mold 110 of FIG. 3 once urethane foam has been injected to form a urethane layer 120 over the felt layer 90. As illustrated in FIG. 4, the sensor sheet 80 includes the felt layer 90 and the urethane layer 120 over the felt layer 90, with the sensor assembly 20 sandwiched between the felt layer 90 and the urethane layer 120.

FIG. 4 also illustrates the sensor insoles 10 being cut from the sensor sheet 80 via a cutting element 12. The cutting element 12 may be any form of cutting device, blade, die, or similar device. The cutting element 12 may be used to cut the sensor sheet 80 around the sensor assembly 20 to form a generally foot-shaped perimeter 100 and thereby forming the sensor insole 10 with the urethane layer 120 surrounding the sensor assembly 20 and over the cut out felt layer 90. The foot-shaped perimeter 100 is not necessarily a particular shape, as long as it may be placed into a shoe or other device to be worn by the user. There may be different sizes of the sensor insoles 10 depending on the size of shoes where the sensor insoles 10 would be used. In one embodiment of the present invention, only five sizes of the sensor insoles 10 are made and all other sizes will be cut or otherwise adapted from these original five sizes.

While FIGS. 2-4 illustrate one embodiment of how the sensor insole 10 (of FIG. 1) may be manufactured, alternative, similar, and equivalent methods may also be used, and such alternative methods of production should be considered within the scope of the present invention.

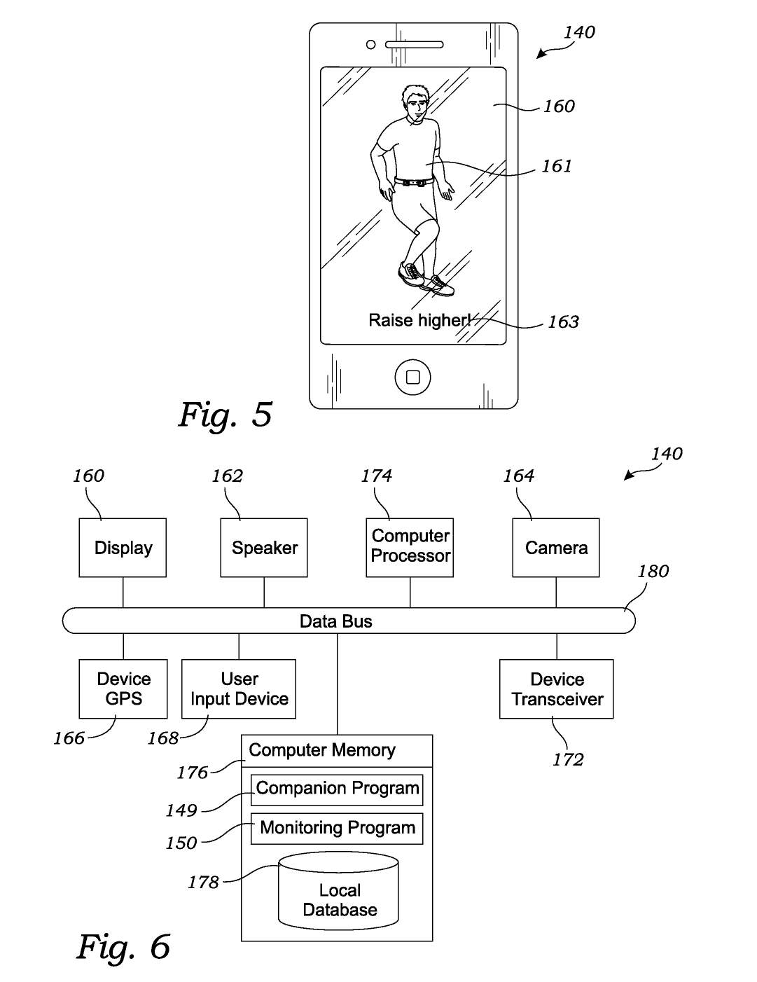

FIG. 5 is a perspective view of one embodiment of a portable electronic device 140 that may be utilized with the sensor insoles 10 (of FIG. 1). As illustrated in FIGS. 5-6, the portable electronic device 140 of this embodiment is a smart phone that includes a monitoring app 150 (discussed in FIG. 6, below) installed thereupon. The application, or “app,” is a computer program that may be downloaded and installed using methods known in the art. The app enables the user to monitor their movement as detected and analyzed by the sensor insoles 10, as illustrated in FIG. 5, and to communicate with the sensor insoles 10 as described in greater detail below to aid in executing proper physical motions. In the discussion of FIGS. 5-6, we will begin with a description of the components of the portable electronic device 140, as they relate to the present invention. Then we will discuss in greater detail the functionality of the monitoring app 150, in one example, an embodiment used for physical therapy, and in another example, an embodiment for being used by a person during running.

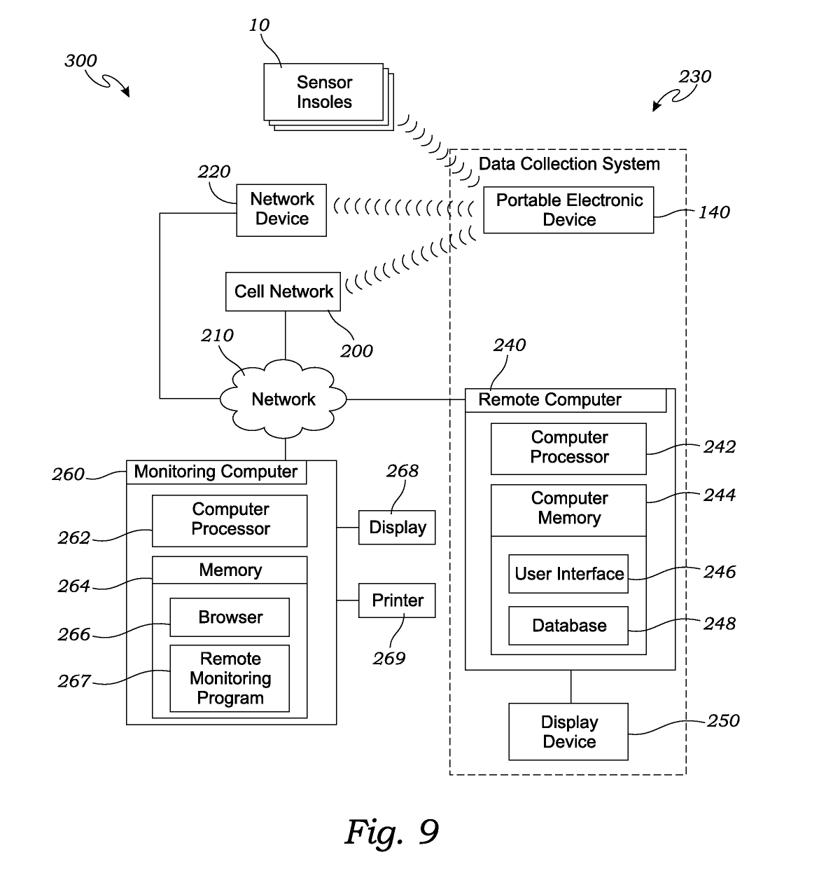

As illustrated in FIG. 5, the monitoring app 150 also monitors a person performing a physical activity such as running, and displays the physical activity in real time (defined to include near-real time, with a slight delay for computer processing, transmission, etc.). The sensor system 300, shown in FIG. 9, includes the sensor insoles 10 and the portable electronic device 140, as discussed above and below in more detail.

In the embodiment of FIG. 5, the monitoring app 150 (of FIG. 6) operably installed on the portable electronic device 140 performs multiple steps. First, a digital model 161 of the person is generated, and the digital model 161 is displayed on the computer display 160 of the portable electronic device 140. Movement of the digital model 161 is displayed, in real time, based upon the data received from the sensor insoles 10 (of FIG. 1), so that the digital model 161 of the person approximates the movement of the person performing the physical activity.

This enables the user to watch himself/herself performing the exercises, to better determine whether they are being performed correctly. The display may also be transmitted to other computer devices, such as a doctor, trainer, caretaker, etc., so that they may monitor the activities and take corrective action if required.

The movement of the digital model 161 may also be compared with a preferred movement model of the monitoring app 150 (of FIG. 6), to determine if the actual movement of the person approximates the preferred movement model, or if correction is needed. Communication with the person, in real time, with corrective instructions 163 may be provided when correction is needed. Corrective instructions 163 may include audio, text, video (e.g., video of the exercise being correctly performed), haptic, and/or any other medium desired to assist the user in performing the exercises such as running (or other activities) correctly.

The system may also provide a script that outlines exactly how the user should run in a physically appropriate manner. For examples countdowns, instructions (e.g., raise leg, lower leg, etc.), which are synchronized with the movements in the video. In this manner, the user is able to perform the run correctly, and receive both instruction and correction, without the requirement of having a personal trainer, which can be expensive. The system is therefore able to deliver superior training, at relatively lower costs, than are available in the prior art.

FIG. 6 is a block diagram of the operable components of the portable electronic device 140 of FIG. 5. The portable electronic device 140 may include various electronic components known in the art for this type of device. In this embodiment, the portable electronic device 140 may include a device display 160, a speaker 162, a camera 164, a device global positioning system (“GPS”), a user input device 168 (e.g., touch screen, keyboard, microphone, and/or other form of input device known in the art), a user output device 170 (such as earbuds, external speakers, and/or other form of output device known in the art), a device transceiver 172 for wireless communication, a computer processor 174, a computer memory 176, the monitoring app 150 operably installed in the computer memory 176, a local database 178 also installed in the computer memory 176, and a data bus 180 interconnecting the aforementioned components. For purposes of this application, the term “transceiver” is defined to include any form of transmitter and/or receiver known in the art, for cellular, WIFI, radio, and/or other form of wireless (or wired) communication known in the art. Obviously, these elements may vary, or may include alternatives known in the art, and such alternative embodiments should be considered within the scope of the claimed invention.

As shown in FIG. 6, the speaker 162, typically integrated into the portable electronic device 140, though the speaker 162 may also be an external speaker, and may give the user audio feedback and instructions during use. The speaker 162 may be any sort of speaker, known by those skilled in the art, capable of transforming electrical signals to auditory output.

Another synergistic use of the monitoring app 150 with common portable electronic devices 140 is that the monitoring app 150 may be continuously calibrated by using the camera 164 of the portable electronic device 140 and common motion capture software. In this instance, if the motion capture determined that both the user's feet were on the ground, but for some reason the monitoring app 150 reported that the user's feet were not at the same level, the position of the user's feet in the monitoring app 150 could be reset to the correct value. The same calibration technique used for position may also be used for the user's velocity and distance travelled based on the number of steps taken, discussed below in greater detail.

The integration of the device GPS 166 and the sensor insoles 10 provides several benefits. First, it may be another potential method of calibration. For example, if the horizontal motion of the sensors (specifically by use of the force sensors 30) have determined that user has travelled a certain distance, agreement can be checked with the device GPS 166 and changes can be made to the data or real-time acquisition programs. The onboard device GPS 166 also increases the safety of the user. If the user was undergoing a strenuous activity and suddenly, and/or for an extended period of time, stopped, the monitoring app 150 may determine that a problem has occurred. The monitoring app 150 could then alert the authorities or others and provide the user's location.

There are many types of user input devices 168 that may be combined for use with the present invention. One type may be the touch-screen capability present in modern smartphones. Here, the user could adjust settings, program routines, select exercises, etc. Various user input devices 168 which may be integrated with present invention, for interfacing with the monitoring app 150 or the sensor insoles 10, should be considered equivalent and within the scope thereof.

The user output devices 170 may be speakers, earbuds, external connections to computers, etc. The user output device 170 is a key component of providing feedback to the user and/or others who may be monitoring the user and is discussed in greater detail below. Various user output devices 170 may be integrated with present invention and should be considered equivalent and within the scope thereof.

The device transceiver 172 may be an integrated wireless transmitter/receiver combination, though a wired connection may be possible or desired in some instances. The device transceiver 172 may be used to communicate with the transceiver 48 on the sensor insole 10, and/or other computers or monitoring devices. Such transceivers are known to those skilled in the art and their equivalents should be considered within the scope of the present invention.

The local database 178 may be included for receiving and storing data temporarily, such as medical programs, therapy routines, logs from earlier use, a predefined distance value, a reference step count, a reference distance ratio, a predefined threshold time, power generated by the user during running, a distance travelled by the user during running, and information about the user; however, this is not required, and all data may be retained in another location if desired.

The above components may be interconnected via the data bus 180, which is a generic term for a conduit of information or electronic signals. There are many possible implementations of the data bus 180 by those skilled in the art, and such implementations should be considered equivalent and within the scope of the present invention.

As illustrated in FIG. 6, the computer memory 176 of the portable electronic device 140 may be used to extend the utility of the portable electronic device 140. In this case, the computer memory of the portable electronic device 140 receives the monitoring app 150 and/or an internet browser for browsing web pages that may include additional medical or training programs. Additional programs may also be included, such as medical diagnostic programs, exercise routines, therapy routines, training programs, and others, some of which are discussed in greater detail below.

We begin a discussion of alternate embodiments of the present invention, by introducing an embodiment where the monitoring app 150 verifies connectivity with the transceiver 48 of the sensor insole 10 and the device transceiver 172. In this embodiment, the monitoring app 150 continually monitors the acquisition of data. Should data acquisition be interrupted, the monitoring app 150 will make a predetermined number of attempts, three for example, to regain connectivity. Should this fail, an alarm or other visual, haptic, or audio cue will be produced, alerting the user to move the portable electronic device 140 closer to the sensor insole 10 in order to regain the data connection.

In the embodiment of FIGS. 5 and 7-8, the monitoring app 150 may be used to generate a graphical user interface on the device display 160 of the portable electronic device 140, as illustrated in FIG. 5, to enable the user to interact with the monitoring app 150. In this embodiment, the graphical user interface may be used to show the user the position of their body, in two or three dimensions, while they are performing the actions required by the instruction program. Also, such instruction may be in the form of audio commands from the speaker 162, visual cues on the monitor of the portable electronic device 140, beeping or other audio cues from the speaker 162 that would indicate pacing or other information, or vibration of the portable electronic device 140. The information given to the user by the monitoring app 150 need not be just instruction, but could also indicate when to start or stop an activity, audio or visual feedback of the results of a completed activity, information on suggested future activities or programs to utilize, or trends of a user's progress in performing various activities.

Using running as one example, the force sensors 30 sense forces applied against the bottom of the foot. With this information, the monitoring app 150 may guide the user as they perform the activity, and reconstruct their motion as it is saved in the computer memory 176. Because the force sensors 30 are located in several places on each foot, the alignment of the foot may be determined. The force sensors 30 may determine if the user is stepping too hard or soft, fast or slow, if their rhythm is correct, if there is a systematic drift during the course of the activity, and more. The monitoring app 150 may also provide feedback and encouragement to the user, telling them how to better perform the activity, giving them the time remaining, or coaxing them to continue even if the monitoring app 150 determines they are becoming fatigued.

In physical therapy it is just as important to not perform an activity incorrectly as it is to perform it correctly. Learning an incorrect way to move may slow the healing process, or even further injure the user. By monitoring the user's motions, the monitoring app 150 can instruct the user to stop if they are performing an activity too wrong, and if the problem cannot be corrected by the feedback provided, to seek the assistance of a medical practitioner before resuming exercises.

In a related embodiment, a companion app 149 may be installed on another instance of the portable electronic device 140, for providing a convenient way of monitoring a patient or user who is using the monitoring app 150, for example a doctor or nurse with the companion app 149 installed on a mobile device, such as a cell phone, laptop computer, tablet computer, etc. The companion app 149 may include the following functionality: the ability to report notifications of the exercise status and sensor insole data, as with the monitoring app 150, the ability to receive text, SMS, or other types of instant messaging or alerts to inform the user of the companion app 150 that the user of the monitoring app 150 has missed an exercise or other scheduled activity such as running, the ability to video the patient performing exercises, with the videos able to be sent to health care providers or others, and the ability to receive notifications from providers or others requesting videos or other data from the patient, practitioner, trainer, or any user of the companion app 149 or monitoring app 150. Other functions of the companion app 149 and their modes of implementation may be added or modified by those skilled in the art, and should be considered equivalent and within the scope of the present invention.

A related feature of the present invention is that it enables, both in real-time and over longer timespans, the user to engage in activities that encourage bilateral equivalence. When an activity is performed, it is often important to not favor one side over another. If a user desires to treat both sides, it is often natural that one side is ‘better’ at an exercise than the other, either due to handedness or prior physical condition. For instance, if one side is stronger than the other, the force sensors 30 may detect greater force applied when the stronger side performs the prescribed action. The monitoring app 150 may detect this favoring, and either explicitly or internal to the routine, instruct the user to perform the actions to bring both sides into equivalent physical condition. Often this requires the analysis of the long-term performance of a user, and here the storage of data on the local database 178 or on the database of a remote computer (shown in FIG. 9) is useful and is described below. With the monitoring app 150 connected to a network (shown in FIG. 9), the data may be monitored in real-time or afterwards by medical practitioners or others. This has the potential for not just the sharing of information with numerous practitioners, but also the monitoring of the user's progress when not on-site, such as therapy performed in the user's home or other location away from the treatment facility.

In yet another embodiment, the monitoring app 150 may contain a mode wherein the monitoring app 150 instructs the force sensors 30 to turn on for only brief periods of time during a longer duration exercise such as running a marathon. This allows data on the user's performance to be sampled throughout the duration of their activity, without the risk of draining the battery 50 as may happen for activities of long duration. Typically the user has entered in the monitoring app 150 an estimate of the duration of their activity, usually measured in hours or fractions thereof. The monitoring app 150 may then pick several times to transition the sensor insoles 10 from a “sleep mode” to a “sprint mode”.

During the “sleep mode” the force sensors 30 are not acquiring data and the battery 50 is putting out minimal power, only enough to maintain telemetry with the monitoring app 150. At the prescribed times, (the “sprint mode”) the monitoring app 150 will instruct the battery 50 to begin a power up cycle, for warming the battery 50 and bringing it to full power. Then the force sensors 30 will be powered and take data for a short span of time, typically about 10 seconds, though the time may be set to be longer or shorter as needed. At the end of the “sprint mode”, data collection ceases and the battery 50 is powered down into “sleep mode” as discussed above. “Sprint mode” may be initiated by voice command, touching the touch-sensitive device display 160 of the portable electronic device 140, or pre-programmed.

In yet another embodiment, the monitoring app 150 may contain a mode useful for acquiring data for a sprinter. In this embodiment, the monitoring app 150 signals the user to begin running. In the case of sprinting, there is a time lag between the start of running and the attainment of the rhythmic full speed run. This occurs when the user is accelerating, getting their stride, etc. To save on memory space, data for some predetermined interval, for example two seconds, is not taken. After the two second delay, data is taken normally and throughout the end of the run. Optionally, data may be taken the entire time in order to capture the start as well, as feedback during that phase may be important to the user's performance. Also, if the user is primarily concerned with monitoring starts, the monitoring app 150 may only run for the first few seconds to record just that portion of the run.

The applications of the present invention go far beyond physical therapy or running For instance the sensor insoles 10 may be used in the training of an athlete such as a martial artist, runner, or bicyclist. Here, the training is very similar to physical therapy, where technique can be monitored with feedback provided to the user and/or trainers. Also a history of the user's progress may be formed for use in charting progress and suggestions for further development.

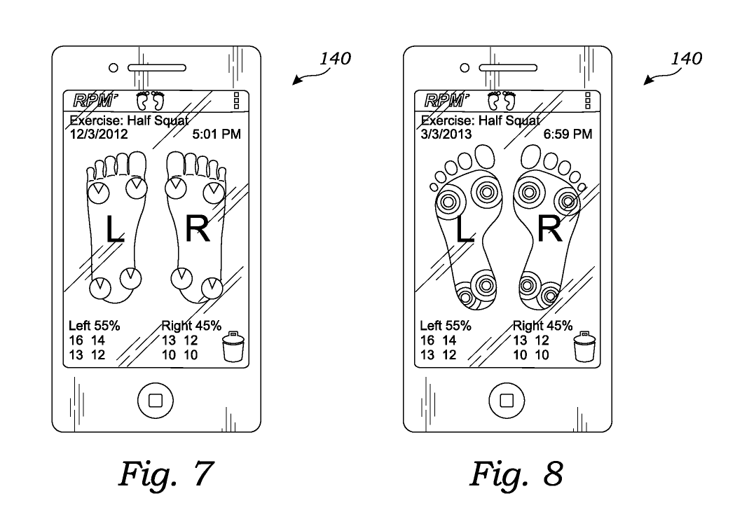

FIG. 7 is a perspective view of the portable electronic device 140 having the monitoring app 150 installed thereupon for monitoring the forces measured by the force sensors 30 in the sensor insoles 10 and illustrating the data in the form of a pie graph. In FIG. 8, the force data may be shown as a pie graph for each of the force sensors 30, containing the percentage of the user's total weight (or applied force) when standing. Alternatively, with a calibrated system, the absolute values may be displayed. The method of display of the data from the sensor insoles 10 may be displayed as shown or in any other method known to those skilled in the art, and a few of those alternate methods are discussed below as alternative embodiments.

FIG. 8 is a perspective view of the portable electronic device 140 having the monitoring app installed thereupon for monitoring the forces measured by the force sensors 30 in the sensor insoles 10 and illustrating the data in the form of a contour plot. FIG. 8 shows an alternate embodiment of the output of the monitoring app 150 as shown on the device display 160 of the portable electronic device 140. Here, the device display 160 shows a contour map of the intensity of the applied force, at the position of the force sensors 30 on the user's foot. In another embodiment, the displayed image may be a heat or intensity map, with the colors corresponding to surfaces of constant force. Additionally the monitoring app 150 may contain an interpolation program, using methods known to those skilled in the art, to provide a more detailed mapping of the force on the bottom of the foot, which may be helpful for medical applications, in particular. Additional numbers of the force sensors 30 may be placed in the sensor insole 10 to increase the accuracy of the interpolation.

The sensor system 300 (shown in FIG. 9) may be used for monitoring and reporting power generated by a person performing an exercise such as running The sensor system 300 comprises the sensor insole 10 (such as is shown in FIG. 1), and the portable electronic device 140 (shown in FIG. 6). In this embodiment, the monitoring app 150 (shown in FIG. 6) is in the form of a power measurement program operably installed in the computer memory 176 of the portable electronic device 140.

As shown in FIGS. 1, 6, and 9, the power measurement program 150 receives data from the force sensors 30 to determine a force generated by the person via the substrate layer of the sensor insole. The power can then be calculated based upon the data received including force generated by the running person, distance travelled by the person, and the taken time, and the power may then be outputted to and displayed on the computer display 160 of the portable electronic device 140, as discussed in greater detail below.

For purposes of this application, the terminology of computing “power” and displaying “power” is hereby defined to include any particular form of power or equivalent measure. This may include an instantaneous measurement, an average over time, peak power, and average peak power, to name a few.

FIG. 9 is a block diagram of one embodiment of a sensor system 300 that includes the portable electronic device 140, a monitoring computer 260, and a remote computer 240 for monitoring the sensor insole 10 and storing data. The sensor insoles 10, in the present embodiment, are operably connected (e.g., wirelessly) to the portable electronic device 140, such as via BLUETOOTH® or similar protocol.

In this embodiment, wherein the portable electronic device 140 is a cellular telephone, the portable electronic device 140 also streams data via a cellular network 200 (and/or another network 210, such as the Internet, or any form of local area network (“LAN”) or a wireless network, to the other computers 260 and/or 240. Alternatively, in another embodiment, the portable electronic device 140 may communicate with the network 210 through a network device 220 such as a wireless transceiver or router. Here we consider two computers in the present embodiment of the invention, the remote computer 240 and the monitoring computer 260.

The remote computer 240 has a computer processor 242, a computer memory 244, a user interface 246 operably installed in the computer memory 244, a database 248 operably installed in the computer memory 244, and a remote display 250. The remote computer 240 functions primarily as a repository of data taken during the user's activity such as running Data stored on the remote computer 240 may be accessed via the network 210 by other computers, or viewed locally using the remote display 250.

The monitoring computer 260 has a computer processor 262, a computer memory 264, a browser 266 operably installed in the computer memory 264, and a monitoring program 267 operably installed in the computer memory 264. Also, the computer may be connected to a monitoring display 268 for viewing the data and/or the output of the monitoring program 267, and have a printer 269 for printing physical copies of the same. The browser 266 may be a typical internet browser or other graphical user interface (“GUI”) that may allow communication over the internet to the patient, other health care practitioners, or trainers. The monitoring program 267 interprets the results of the data sent by the monitoring app 150 and provides analysis and reports to the user of the monitoring computer 260. The monitoring program 267 provides information not included in the monitoring app 150, for example diagnosis of conditions and suggestions for treatment, or comparison of results with other patients either in real-time or by accessing the database 248 of the remote computer 240.

One embodiment of the sensor system 300 includes providing the various components, particularly the force sensors 30, a unique address programmed therein for identification. The sensor system 300 includes a data collection system 230 for simultaneously monitoring both the first and second locations and, in addition to any other number of locations that may be desired, around the world.

In this embodiment, the data collection system 230 may include a cell phone (such as is shown in FIG. 5), and the remote computer 240 for simultaneously monitoring both the first location and a second location. In alternative embodiment, any one of these elements, or combinations thereof, may be used, in addition to any additional computer devices for tracking the data.

In this embodiment, a unique address is stored in each of the various components, and may include an IP address, or any form of unique indicator (e.g., alphanumeric). The address may be stored in the memory 264, or in any other hardware known in the art, and is transmitted with the data so that the data may be associated with the data in a database (e.g., the local database 178 of the portable electronic device 140, or the database 248 of the remote computer 240). This method is discussed in greater detail below.

Data from the various components may then be streamed to the remote computer 240 (or other component of the data collection system 230) for storage in the database 248. For purposes of this application, “streaming data” may be performed in real time, with data being constantly transmitted (e.g., in typical “packets”), or it may be aggregated and sent periodically, or it may be stored and periodically downloaded (e.g., via USB or other connection) and transmitted.

In one embodiment, the data may include force data from the at least one of the force sensors 30. Selected data, such as the force data, may be transmitted in real time, while more complex data, such as the movement data may be stored in the memory 46 until a suitable trigger, such as actuation of a pushbutton, passage of a predetermined period of time, or other trigger (e.g., at the end of an exercise), and then streamed as a single transmission. Transmitting the data in this manner has proven to greatly relieve demands on the sensor insoles 10, which might otherwise make management of the data extremely difficult, especially when large numbers of users are utilizing the system.

In one embodiment, the data may be periodically analyzed by the remote computer 240 (or other suitable computer system) for “alarm conditions” (e.g., information and/or deviations that may be of interest to the user and/or the doctor and/or any other form of administrator). If an alarm condition is detected, a pertinent alert may be sent to the monitoring computer 260, directly to the user (e.g., via text message, email, signal to the portable electronic device 140, etc.), or to any other suitable party. For example, if the user is putting too much force on an injured leg during rehabilitation, or performing the exercise incorrectly, an alert may be sent to the user for immediate action, and/or a message (e.g., training video, etc.) may be sent via email or other method to help the user perform the exercise correctly.

In another embodiment, in which the sensor system 300 is used in an industrial setting, reports may be sent to supervisors to correct incorrect behavior of workers. In the case of monitoring workers compensation recipients, a fraud monitor may be alerted if the recipient is detected acting in a manner inconsistent with their injury (e.g., playing a sport).

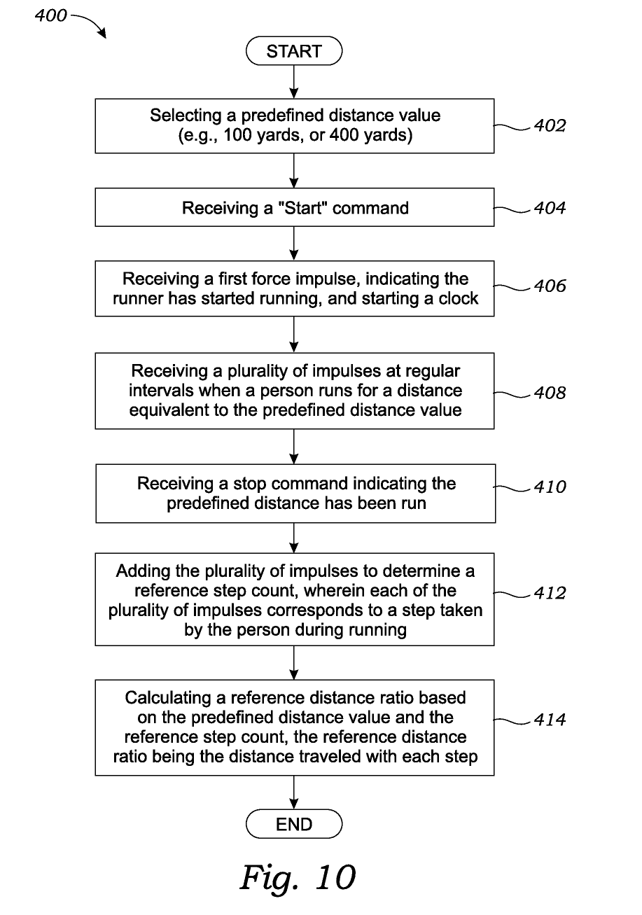

FIG. 10 is a flow diagram illustrating an exemplary method implemented by the portable electronic device 140 of FIGS. 5 and 6 (or other suitable computer device) for calibrating the device 140 for measuring the power generated by the user while running The exemplary method 400 may be described in the general context of computer executable instructions. Generally, computer executable instructions may include routines, programs, objects, components, data structures, procedures, modules, functions, and the like that perform particular functions or implement particular data types. The computer executable instructions may be stored on a computer readable medium, and installed or embedded in an appropriate device for execution. The order in which the method 400 is described is not intended to be construed as a limitation, and any number of the described method blocks may be combined or otherwise performed in any order to implement the method 400, or an alternate method. Additionally, individual blocks may be deleted from the method 400 without departing from the spirit and scope of the present disclosure described herein. Furthermore, the method 400 may be implemented in any suitable hardware, software, firmware, or combination thereof, that exists in the related art or that is later developed.

The method 400 as described is implemented of the portable electronic device 140; however, those having ordinary skill in the art would understand that the method 400 may be modified appropriately for implementation in a various alternative manners without departing from the scope and spirit of the disclosure. Any computer device desired, including remote computers, and/or various devices carried or worn by the user, may be utilized for calculating and/or displaying the power exerted by the user.

As shown in FIG. 10, at step 402, a predefined distance value is selected. The portable electronic device 140 may receive a predefined distance value from a user via various user input devices 168 in communication with the portable electronic device 140 (e.g., 100 yards, 400 yards, etc.). In this embodiment, the predefined distance value may be selected from a list of typical distance values, which may be preset in the portable electronic device 140. In alternative embodiments, one or more distance values may be presented to the user for selection based on various attributes of the user, e.g., age, health condition, difficulty level, etc. stored in the local database 178. In some other embodiments, the user may input a distance that they desire, or the portable electronic device 140 may automatically or randomly generate a single predefined distance value based on user preferences or user profiles stored in the local database 178. Such preferences or profiles may include, but not limited to, health data, exercise data, and availability data for one or more users.

At step 404, the portable electronic device 140 may receive a start command from one of the input devices 168 to listen for an impulse being received from the force sensors 30. This may be initiated by pressing a button on the portable electronic device 140 of FIG. 5, or taking some other form of action (e.g., shaking the portable electronic device 140, voice command, etc). This command indicates that the user is ready to start. In some embodiments, this action alone starts the below-described process.

In another embodiment, as shown in FIG. 10, the actual commencement of running is what finally initiations the below-described method. At step 406, the portable electronic device 140 receives a first impulse indicating that a person has started running Upon receiving the first impulse, the portable electronic device 140 may initiate a clock timer (not shown) that calculates the time elapsed since the first impulse is received until a stop command is received by the portable electronic device 140.

At step 408, the portable electronic device 140 may receive multiple impulses at regular intervals based on the person beginning to run or when the user actually runs a distance equivalent to the predefined distance value. If the impulses are not received at generally regular intervals, this may indicate a hardware error, or a problem with the user's running, and this may generate an error message and require that the calibrating run be made again. Those skilled in the art may determine a range of time that is considered “generally regular intervals”, which enables changes and irregularities in a runner's gait, while also being able to determine if there are substantial errors that may require the calibration to be performed again.

During the calibration run, the portable electronic device 140 receives the impulses, and uses these impulses to determine how many steps are taking during the course of the calibration run. It may also track the time required for the run, but this is not required for the calibration process.

At step 410, the portable electronic device 140 receives a stop command from the user through any of the input devices 168. The stop command may include any form of actions discussed above (e.g., pressing a button, shaking the portable electronic device 140, etc.) to indicate that the user has completed running the predefined distance. In some embodiments, the stop command may be made by an alternative form of physical activity, e.g., jumping, skipping, etc., which may be detected by the force sensors 30 and used to provide the stop command. The portable electronic device 140 may accordingly stop the clock timer and determine the time elapsed since the first impulse was received from the clock timer.

At step 412, the portable electronic device 140 adds the number of impulses received during the calibration run, which may be used to determine a reference step count. Each of the received impulses may correspond to a step being taken by the user while running so as to exert actual or estimated force on the force sensors 30.

At step 414, the portable electronic device 140 may be configured to calculate a reference distance ratio that may refer to a ratio of the selected predefined distance value and the reference step count. The reference distance ratio may correspond to an actual or estimated distance travelled by the user with each step indicated by each impulse received by the portable electronic device 140.

The method illustrated in FIG. 10 may be used to determine approximately the distance traveled with each step, a value which is used in the below-described process for calculating power. While this one method is described in detail, one having ordinary skill in the art may determine alternative methods for determining the distance per step, and such alternatives are within the scope of the present invention. Furthermore, the person controlling or operating the portable electronic device 140 may be different from the person running the distance equivalent to the predefined distance value.

In some embodiments, this calibration process may be performed more than once, to confirm that substantially similar results are achieved. The results may be averaged, or faulty results may be discarded, using techniques known in the art.

FIG. 11 is a flow diagram illustrating an exemplary method implemented by the portable electronic device 140 of FIG. 9 (or other suitable computer device) for measuring power generated by the user while running, using the calibration information generated by the method of FIG. 10 (i.e., the distance of each stride of the runner). The exemplary method 500 may be described in the general context of computer executable instructions. Generally, computer executable instructions may include routines, programs, objects, components, data structures, procedures, modules, functions, and the like that perform particular functions or implement particular data types. The computer executable instructions may be stored on a computer readable medium, and installed or embedded in an appropriate device for execution. The order in which the method 500 is described is not intended to be construed as a limitation, and any number of the described method blocks may be combined or otherwise performed in any order to implement the method 500, or an alternate method. Additionally, individual blocks may be deleted from the method 500 without departing from the spirit and scope of the present disclosure described herein. Furthermore, the method 500 may be implemented in any suitable hardware, software, firmware, or combination thereof, that exists in the related art or that is later developed.

The method 500 describes one method of implementing the invention using the sensor insole 10 and the portable electronic device 140, as described above, or via equivalent devices. Those having ordinary skill in the art would understand that the method 500 may be modified appropriately for implementation in a various manners without departing from the scope and spirit of the disclosure, and such alternatives should be considered within the scope of the present invention.

As shown in FIG. 11, at step 502, a start command is received. As discussed above, the portable electronic device 140 (or other device) may receive a start command from any of the user input devices 168, in any manner known in the art. Upon receiving the start command, the portable electronic device 140 may also await an impulse being received from the force sensors 30 (or not, depending upon the desires of the system designer).

At step 504, the portable electronic device 140 receives force data from the force sensors 30 via the transmitter 48 when the user is in motion such as when the user is running The force data may include one or more impulses generated by one or more force sensors 30 in response to the user exerting a force on respective force sensors 30 during running Upon receiving or detecting a first impulse from at least one of the force sensors 30, the portable electronic device 140 may initiate a clock 47 (as shown in FIG. 1) configured to begin calculating a time duration since the receipt of the start command (e.g., button pressed, first impulse, etc.) to the stop command. The clock 47 may itself provide the start and stop commands (e.g., for every selected number of seconds, etc.). Once the user starts a run, selected periods of time may be analyzed and a power reported.

The portable electronic device 140 adds the number of impulses received during this time period, and, further, the portable electronic device 140 may be configured to calculate the force exerted by each impulse received from one or more of the force sensors 30 based on equation 1.

$$F = \frac{I}{t} \qquad ( 1 )$$

In equation 1, ‘F’ refers to a force exerted by a foot on one or more of the force sensors 30 during running; ‘I’ refers to an impulse generated by based on that exerted force; and ‘t’ refers to a time duration for which that impulse is received by the portable electronic device 140.

At step 506, the portable electronic device 140 checks whether or not it has received a stop command through any of the input devices 168. If no stop command is received, the portable electronic device 140 moves back to the step 504 and repeats steps 504 and 506; else the portable electronic device 140 moves ahead to execute step 508. In some embodiments, the portable electronic device 140 may be configured to generate a stop command in case no impulse is received for a predefined time threshold. In some other embodiments, the stop command may refer to any alternative form of physical activity, e.g., jumping, skipping, etc., which may be detected by the force sensors 30 and used to provide the stop command.

As shown in FIG. 11, at step 508, the portable electronic device 140 may calculate a total distance travelled by a running user based on a product of the calculated total number of impulses received at step 504 and the reference distance ratio, which may be predefined into the portable electronic device 140 during calibration. In some embodiments, the insole 10 does not include a GPS or accelerometers, although in alternative embodiments these may be included as well.

At step 510, the portable electronic device 140 may determine a total time from the timer during which the impulses were received. Such total time may refer to a time duration between an instant at which the timer was initiate and an instant when the timer was stopped by the portable electronic device 140. In other embodiments, it may be selected time periods during a run (to determine the power at these particular points in the run). At step 512, the portable electronic device 140 may calculate a cumulative force generated by the total number of impulses by adding a force for each received impulse that was calculated previously at step 504.

At step 514, the portable electronic device 140 may calculate the power generated by the user while being in the running state based on equation 2.

$$P = F * \frac{d}{t} \qquad ( 2 )$$

In equation 2, power (P) may be work (a force applied over a distance) done in a period of time; a cumulative force (F) exerted through a foot during running (or, walking or other activity) that results in a movement in a human, animal or machine (illustratively a plurality of propulsive impulses may be measured as discussed in steps 504 and 512); distance (d) may be traveled by the foot as a consequence of the aforementioned force (F). This distance is illustratively measured based on a product of the total number of impulses received and the reference distance ratio as discussed in step 508, and time (t) may be calculated inclusively between a first impulse received and a last impulse received, as discussed in step 510, to calculate power generated by a running entity such as a human, animal or machine. In some embodiments, the portable electronic device 140 may calculate the power periodically based on the clock timer being configured to trigger the stop command after a predefined duration (e.g., sixty seconds).

At step 516, the portable electronic device 140 may report the calculated power to a user by displaying it on its display device 160 or may communicate the calculated power to other networked devices such as a monitoring computer 260 or a remote computer 240 for display on respective display devices 268 and 250.

The computer or computers used in the sensor system may be any form of computers or computers, servers, or networks known in the art. As used in this application, the terms computer, processor, memory, and other computer related components, are hereby expressly defined to include any arrangement of computer(s), processor(s), memory device or devices, and/or computer components, either as a single unit or operably connected and/or networked across multiple computers (or distributed computer components), to perform the functions described herein.

The exemplary embodiments described herein detail for illustrative purposes are subject to many variations of structure and design. It should be emphasized, however that the present invention is not limited to particular method of manufacturing sensor insoles as shown and described. Rather, the principles of the present invention can be used with a variety of methods of manufacturing sensor insoles. It is understood that various omissions, substitutions of equivalents are contemplated as circumstances may suggest or render expedient, but the present invention is intended to cover the application or implementation without departing from the spirit or scope of the claims.

As used in this application, the words “a,” “an,” and “one” are defined to include one or more of the referenced item unless specifically stated otherwise. Also, the terms “have,” “include,” “contain,” and similar terms are defined to mean “comprising” unless specifically stated otherwise. The term ‘shoes’ or ‘footwear’ may have been used above interchangeably and refer to convey the same meaning. The term “activity” as used in this application refers to any activity that the user of the present invention may be undertaking, whether it is exercise, training, physical therapy, or routine activities. Also, pressure and force may be used interchangeably as pressure is simply a scalar quantity that relates the applied force to a known surface area. Furthermore, the terminology used in the specification provided above is hereby defined to include similar and/or equivalent terms, and/or alternative embodiments that would be considered obvious to one skilled in the art given the teachings of the present patent application.

What is claimed is:

1. A method for measuring power generated by a person during running, wherein at least one of the person's feet is in mechanical communication with a pair of sensor insoles, each having a plurality of force sensors, the method comprising the steps of:
storing a reference distance ratio for the person that is an approximate distance travelled for each step taken by that person;
receiving force data from the plurality of force sensors for a designated period, the force data including a number of impulses received that correspond to the number of steps taken by the person, and a force for each of the impulses;
calculating a distance run during the designated period based on a product of the reference distance ratio and the total number of impulses received in the force data;
calculating a total force during the designated period based on a sum of the force measured in all of the impulses in the force data;
determining time elapsed for the designated period;
calculating power during the designated period based on the calculated distance, the elapsed time, and the total force determined; and
reporting the power calculated for the designated period.

2. The method of claim 1, wherein the designated period is the time between the receipt of a start command and the receipt of a stop command.

3. The method of claim 2, wherein the start command and the stop command are generated by a portable electronic device.

4. The method of claim 1, wherein the designated period is a predetermined segment of time that is between the receipt of a start command and the receipt of a stop command.

5. The method of claim 1, wherein the reference distance ratio is determined by running a predefined distance and determining the total number of impulses received, each impulse indicating a step taken by the person to run across a distance equivalent to the predefined distance value.

6. A method for calibrating a portable electronic device capable of measuring power generated by a person during running, wherein at least one of the person's feet is in mechanical communication with a pair of sensor insoles, each having a plurality of force sensors and a transmitter for transmitting data from the plurality of force sensors, the method comprising the steps of:
receiving, via a portable electronic device, a predefined distance value;
receiving, with a transceiver on the portable electronic device, a plurality of impulses at regular intervals from the plurality of force sensors via the transmitter when the person runs for a distance equivalent to the predefined distance value;
calculating, with a computer processor on the portable electronic device, a reference step count in response to the plurality of impulses, wherein each of the plurality of impulses corresponds to a step taken by the person during running; and
calculating, with the computer processor, a reference distance ratio based on the predefined distance value and the reference step count.

7. The method of claim 6, wherein the reference distance ratio is calculated by adding the number of the plurality of impulses.

8. A method for measuring power generated by a person during running, the method comprising the steps of:
providing a pair of sensor insoles, each having a plurality of force sensors;
providing a portable electronic device having a computer processor and a computer memory;
operably positioning each of the sensor insoles under one of the feet of the person;
calibrating a portable electronic device by determining a reference distance ratio for the person that is an approximate distance travelled for each step taken by that person, the calibration comprising the following steps:
receiving, with a user input device on the portable electronic device, a predefined distance value;
receiving, with a transceiver on the portable electronic device, a plurality of impulses at regular intervals from the plurality of force sensors via the transmitter when the person runs for a distance equivalent to the predefined distance value;
calculating, with a computer processor on the portable electronic device, a reference step count in response to the plurality of impulses, wherein each of the plurality of impulses corresponds to a step taken by the person during running; and
calculating, with the computer processor, a reference distance ratio by dividing the predefined distance value by the reference step count;
following calibration, running while the portable electronic device receives force data from the plurality of force sensors for a designated period, the force data including a number of impulses received that correspond to the number of steps taken by the person, and a force for each of the impulses;
calculating a distance run during the designated period based on a product of the reference distance ratio and the total number of impulses received in the force data;
calculating a total force during the designated period based on a sum of the force measured in all of the impulses in the force data;
determining time elapsed for the designated period;
calculating power during the designated period based on the calculated distance, the elapsed time, and the total force determined; and
reporting the power calculated for the designated period.
